(19) United States
(12) Patent Application Publication
(10) Pub. No.: US 2021/0401474 A1
(43) Pub. Date: Dec. 30, 2021

(54) FRACTURE FIXATION SYSTEM

(71) Applicant: Daniel CHAN, Macon, GA (US)

(72) Inventor: Daniel CHAN, Macon, GA (US)

(21) Appl. No.: 17/292,205

(22) PCT Filed: Nov. 8, 2019
(86) PCT No.: PCT/US2019/060505

§ 371 Date: May 7, 2021

Related U.S. Application Data
(60) Provisional application No. 62/758,456, filed on Nov. 9, 2018.

(51) Int. Cl. A61B 17/80; A61B 17/82

(52) U.S. Cl. CPC A61B 17/8014; A61B 17/8605; A61B 17/82; A61B 17/8057

(57) ABSTRACT

In various implementations, a fracture fixation system includes a plate, at least two distal screws, at least one fastener, and at least one wire. The plate has a proximal end, a distal end, and a longitudinal axis that extends between the proximal and distal ends. The plate also includes a distal end portion adjacent the distal end that defines at least two circular openings and an intermediate portion disposed between the proximal end portion and distal end portion. The intermediate portion defines at least one axially oriented elongated slot. The distal screws engage the respective circular openings and a distal end of the bone below a fracture of the bone. The fastener is slidably coupled to the axially oriented elongated slot to prevent movement laterally, rotationally, or angularly of the bone relative to the plate while allowing the proximal and distal portions of the bone to compress the fracture.

### CROSS REFERENCE TO RELATED APPLICATIONS

This application is a national stage application filed under 35 U.S.C. § 371 of PCT/US2019/060505 filed Nov. 8, 2019, which claims priority to U.S. Patent Application No. 62/758,456, entitled “Fracture Fixation System,” filed Nov. 9, 2018, the content of which is herein incorporated by reference in its entirety.

### BACKGROUND

Current femur fracture fixation systems include a plate and plurality of screws for affixing the plate against the femur. One or more screws extend through the plate and bone on a first side of the fracture, and one or more screws extend through the plate and the bone on a second side of the fracture. The screws extend through openings defined in the plate that are generally sized to prevent movement of the plate relative to the bone. Conventional plate constructs prevent the proximal and distal portions of the bone to move relative to each other along the plate to compress the fracture.

Thus, there is a need in the art for a fixation system that allows for axial compression of the fractured portion of the bone while preventing the portion of the bone above the fracture from shifting laterally, rotationally, or angularly relative to the portion of the bone below the fracture.

### SUMMARY

Various implementations include a fracture fixation system. The fracture fixation system includes a plate, at least two distal screws, and at least one fastener. The plate has a proximal end, a distal end, and a longitudinal axis that extends between the proximal end and the distal end. The plate further includes a distal end portion adjacent the distal end that defines at least two circular openings and an intermediate portion disposed between the proximal end and the distal end portion. The intermediate portion defines at least one axially oriented elongated slot. The at least two distal screws are for engaging the respective circular openings and a distal end of a bone below a fracture of the bone. The at least one fastener is slidably coupled to the axially oriented elongated slot. The fastener laterally secures the plate adjacent the bone. A distal end of the elongated slot is defined to be disposed above the fracture of the bone when the plate is secured to the bone with the distal screws.

In some implementations, the fastener includes an eyelet slidably coupled to the axially oriented elongated slot and at least one wire for threading through the eyelet and securing around the plate and the bone. In some implementations, the at least one axially oriented elongated slot includes a first slot and a second slot. The slots are axially aligned. The eyelet is slidably coupled to the first slot. The system further includes an intermediate screw for engaging the second slot and a portion of the bone above the fracture of the bone. The intermediate screw is axially slidable within the second slot. In some implementations, the first and second slots define longitudinal grooves that engage threads on the eyelet and the intermediate screw, respectively. In some implementations, the intermediate screw is a bicortical or unicortical screw. In some implementations, the distal screws are bicortical locking screws.

In some implementations, the grooves are oriented at a pitch of greater than 0° relative to the longitudinal axis of the plate for preventing lateral movement of the eyelet and the intermediate screw from the first and second elongated slots, respectively, and allowing the eyelet and the intermediate screw to slide within the first and second elongated slots, respectively.

In some implementations, the first slot is closer to the proximal end of the plate than the second slot. In some implementations, the second slot is closer to the proximal end of the plate than the first slot.

In some implementations, the fastener includes an intermediate screw for engaging the slot and a portion of the bone above the fracture of the bone. The intermediate screw is axially slidable within the slot.

In some implementations, a longitudinal length of the slot is between 2 mm and 10 mm. In some implementations, the longitudinal length of the slot is 7 mm.

In some implementations, the distal screws are bicortical locking screws.

In some implementations, the bone is a femur.

Various other implementations include a fracture fixation method. The fracture fixation method includes disposing a plate against a fractured bone. The plate has a proximal end, a distal end, and a longitudinal axis that extends between the proximal end and the distal end. The plate further includes a distal end portion adjacent the distal end that defines at least two circular openings and an intermediate portion disposed between the proximal end and the distal end portion. The intermediate portion defines at least one axially oriented elongated slot. The fracture fixation method further includes engaging at least two distal screws into the respective circular openings and a distal end of the bone below a fracture of the bone and slidably coupling at least one fastener to the axially oriented elongated slot. The at least one fastener laterally secures the plate adjacent the bone. A distal end of the elongated slot is disposed above the fracture of the bone.

In some implementations, slidably coupling the at least one fastener includes slidably coupling an eyelet to the axially oriented elongated slot, threading at least one wire through the eyelet, and securing the wire around the plate and the bone. In some implementations, the at least one axially oriented elongated slot includes a first slot and a second slot. The slots are axially aligned. The eyelet is slidably coupled to the first slot. The method further includes engaging an intermediate screw through the second slot and a portion of the bone above the fracture of the bone. The intermediate screw is axially slidable within the second slot. In some implementations, the first and second slots define longitudinal grooves that engage threads on the eyelet and the intermediate screw, respectively. In some implementations, the intermediate screw is a bicortical or unicortical screw. In some implementations, the distal screws are bicortical locking screws.

In some implementations, the grooves are oriented at a pitch of between 1° and 2° relative to the longitudinal axis of the plate.

In some implementations, the first slot is closer to the proximal end of the plate than the second slot. In some implementations, the second slot is closer to the proximal end of the plate than the first slot.

In some implementations, a longitudinal length of the slot is between 2 mm and 10 mm. In some implementations, the longitudinal length of the slot is 7 mm.

In some implementations, the distal screws are bicortical locking screws.

In some implementations, slidably coupling at least one fastener to the axially oriented elongated slot includes engaging an intermediate screw through the slot and a portion of the bone above the fracture of the bone. The intermediate screw is axially slidable within the slot.

In some implementations, the bone is a femur.

### BRIEF DESCRIPTION OF THE DRAWINGS

FIG. 1 illustrates a perspective view of a femur fracture fixation system according to one implementation.

FIG. 2A illustrates a cross sectional view of the femur fracture fixation system as taken through the A-A line shown in FIG. 1.

FIG. 2B illustrates a close-up view of elongated slot 106a as shown in FIG. 2A. Because the slots 106b, 106c are identical to slot 106a, they are not shown in a close-up view.

FIG. 3 illustrates a cross sectional view of the femur fracture fixation system as taken through the B-B line shown in FIG. 1.

### DETAILED DESCRIPTION

Various implementations include fracture fixation systems and methods. In some implementations, a fracture fixation system includes a plate, at least two distal screws, at least one fastener, and at least one wire. The plate has a proximal end, a distal end, and a longitudinal axis that extends between the proximal end and the distal end. The plate also includes a distal end portion adjacent the distal end that defines at least two circular openings and an intermediate portion disposed between the proximal end and the distal end portion. The intermediate portion defines at least one axially oriented elongated slot. The distal screws are for engaging the respective circular openings and a distal end of the bone below a fracture of the bone. The fastener is slidably coupled to the axially oriented elongated slot and laterally secures the plate adjacent the bone. A distal end of the elongated slot is defined to be disposed above the fracture of the femur when the plate is secured to the femur with the distal screws. In some implementations, the fracture fixation system is a femoral fracture fixation system, the plate is a femoral plate, and the bone is the femur. However, in other implementations, the fracture fixation system can be used to assist with healing of other bones.

In some implementations, a femur fracture fixation method includes disposing a plate against a fractured bone, such as the plate described above, engaging distal screws into the respective circular openings and a distal end of the bone below a fracture of the bone, and slidably coupling at least one fastener to the axially oriented elongated slot and laterally securing the plate adjacent the bone. A distal end of the elongated slot is disposed above the fracture of the bone.

The system and method provide a plate that prevents laterally, rotationally, or angularly shifting of the portions of the bone above and below the fracture relative to each other but allows the portions of the bone on either side of the fracture to compress against the fracture due to some amount of axial loading by the patient. This compression causes callus formation and bone healing. Various implementations may be used to prevent above mentioned shifting and promote healing of fractures in the femur and/or other bones.

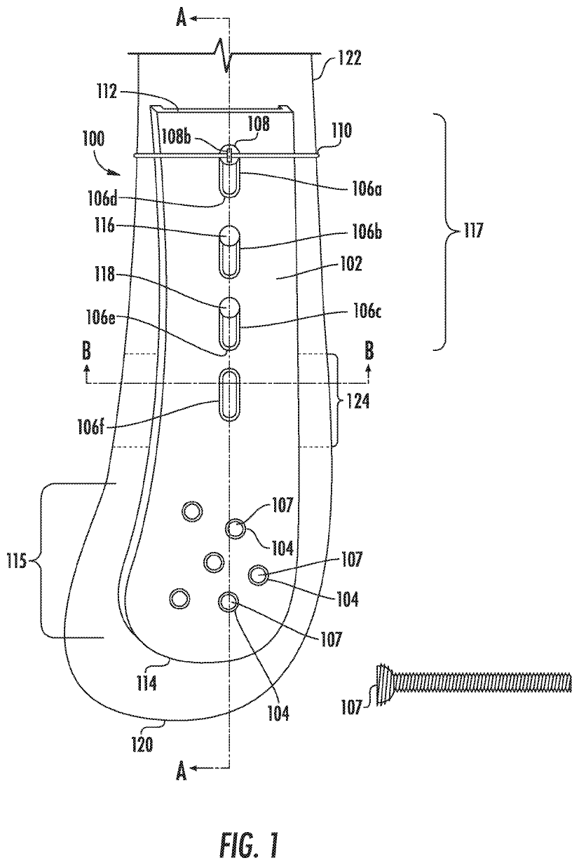

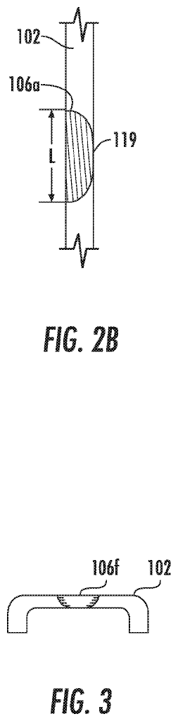

For example, FIGS. 1-3 illustrate a femur fracture fixation system 100 according to one implementation. The system 100 includes a plate 102, one or more distal screws 107, an eyelet 108, a wire 110 (e.g., cerclage wire), and intermediate screws 116, 118. The plate 102 has a proximal end 112, a distal end 114, and a longitudinal axis A-A that extends between the proximal end 112 and the distal end 114. The plate 102 further comprises a distal end portion 115 adjacent the distal end 114 that defines two or more circular openings 104 and an intermediate portion 117 disposed between the proximal end 112 and the distal end portion 115. The intermediate portion 117 defines axially oriented elongated slots 106a, 106b, 106c, 106f. The slots 106a, 106b, 106c, 106f are axially aligned, the slot 106a is closest to the proximal end 112 of the plate 102, and the slots 106b, 106c are defined between slots 106a and 106f. Although four slots are defined in the implementation shown in FIG. 1, one or more slots may be defined in other implementations. In some implementations, the plate 102 is made from stainless steel. However, in other implementations, the plate may be made from other materials, such as titanium.

Distal screws 107 engage the respective circular openings 104 adjacent a distal end 120 of the femur bone 122 below a fracture 124 of the femur 122. One or more fasteners engage one or more respective slots 106a, 106b, 106c, 106f above the fracture 124 of the femur 122. For example, in FIGS. 1-3, the fastener engaged in slot 106a includes eyelet 108 and wire 110. In particular, eyelet 108 is slidably coupled to the axially oriented elongated slot 106a, and the wire 110 is threaded through the eyelet 108 and secured around the plate 102 and femur bone 122. A distal end 106d of the elongated slot 106a is defined to be disposed above the fracture 124 of the femur bone 122 when the plate 102 is secured to the femur bone 122 with the distal screws 107. In addition, fasteners engaged in slots 106b and 106c include intermediate screws 116, 118. The intermediate screws 116, 118 are engaged through the second slot 106b and the third slot 106c, respectively, and into the femur bone 122. And, the intermediate screws 116, 118 are axially slidable within the slots 106b, 106c. The distal end 106e of the third slot 106c is disposed above the fracture 124 of the femur bone 122. In the example shown in FIGS. 1-3, slot 106f is disposed such that the fracture zone 124 is directly behind slot 106f, and no fastener is engaged within slot 106f. Fasteners may or may not be placed in slots where the fracture zone 124 is directly behind the slots. In some implementations, no fastener may be engaged within one or more slots defined by the plate.

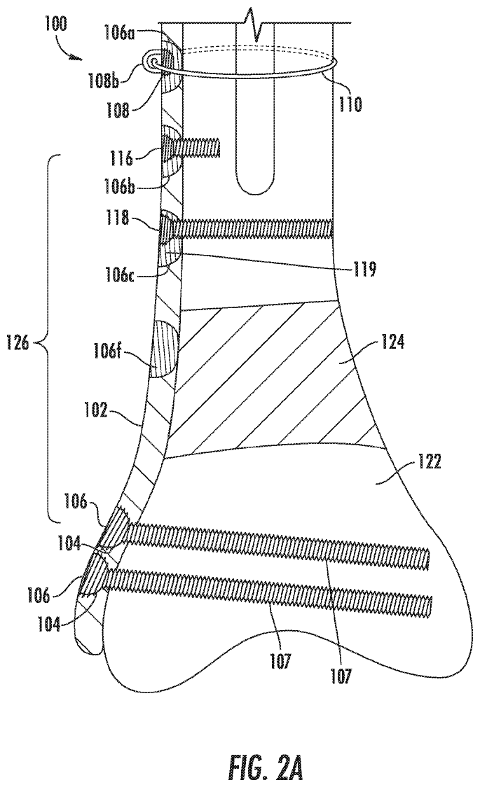

As shown in FIGS. 2A and 2B, the elongated slots 106a, 106b, 106c, 106f define longitudinal grooves 119. The eyelet 108 has an engagement portion 108b that has threads. The threads of the engagement portion 108b of the eyelet 108 and of the screws 116, 118 engage with the grooves 119 and are axially slidable within the grooves 119. However, the grooves 119 prevent the screws 116, 118 from unintentionally moving laterally relative to the bone 122 and plate 102. The grooves 119 are defined on faces of opposing walls of the slots 106a, 106b, 106c, 106f. In some implementations, the grooves 119 are oriented at a pitch of greater than 0° (e.g., 1° to 2°) relative to the axis A-A extending through the slots 106a, 106b, 106c, 106f for preventing lateral movement of the eyelet and the intermediate screw from the first and second elongated slots, respectively, and allowing the eyelet and the intermediate screw to slide within the first and second elongated slots, respectively.

Furthermore, as shown in FIG. 2A, the intermediate screw 116 is a unicortical screw, and the intermediate screw 118 is a bicortical screw. However, in other implementations, either screw 116, 118 may be unicortical or bicortical. For example, in some implementations, bicortical screws may be used when there are no implants or other structure within the femur bone that would interfere with the use of the bicortical screw, and unicortical screws may be used when there are implants or other structures within the femur bone that would interfere with the use of a bicortical screw. The distal screws 107 are bicortical locking screws, but in other implementations, the distal screws may be unicortical locking screws or a combination of bicortical and unicortical locking screws.

Although the eyelet 108 is engaged in slot 106a in FIGS. 1-2A, in some implementations, the eyelet 108 may be engaged in one of the other slots 106b, 106c, 106f, and an intermediate screw may be engaged in slot 106a. And, in some implementations, one or more intermediate screws may be used instead of the eyelet and wire.

The slots 106a, 106b, 106c, 106f have a longitudinal length L as measured between a proximal end and a distal end of each slot in the direction of the longitudinal axis A-A of between 2 mm and 10 mm. For example, in some implementations, the longitudinal length L of the slot is 7 mm.

To fix a femur fracture 124 using the system 100 described above, the plate 102 is disposed against a fractured femur 122. The distal end portion 115 of the plate 102 is disposed below the fracture 124, and the intermediate portion 117 (or a portion thereof) is disposed above the fracture 124. The longitudinal axis A-A of the plate is aligned to be generally parallel with a longitudinal axis of the femur bone 122 being fixed. At least two distal screws 107 are engaged into the respective circular openings 104 adjacent the distal end 120 of the femur 122 below the fracture 124. The wire 110 is thread through the eyelet 108 that is slidably coupled to the axially oriented elongated slot 106a and is secured around the plate 102 and the femur 122. A distal end 106d of the elongated slot 106a is disposed above the fracture 124 of the femur 122. Intermediate screws 116, 118 are engaged in slots 106b, 106c respectively. Because the eyelet 108 and intermediate screws 116, 118 can move within the respective slots 106a, 106b, 106c when the proximal end of the femur 122 is compressed (e.g., due to weight bearing activity by the patient), an area 126 of the femur 122 including the fracture 124 is symmetrically compressed, which allows calluses to form and the bone 122 to heal more quickly.

The terminology used herein is for the purpose of describing particular implementations only and is not intended to be limiting of the claims. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description has been presented for purposes of illustration but is not intended to be exhaustive or limited to the implementations in the form disclosed. Many modifications and variations will be apparent to those of ordinary skill in the art without departing from the scope and spirit of the claims. The implementations were chosen and described to best explain the principles of the claims and the practical application, and to enable others of ordinary skill in the art to understand the claims for various implementations with various modifications as are suited to the particular use contemplated.

1. A fracture fixation system comprising:
a plate having a proximal end, a distal end, and a longitudinal axis that extends between the proximal end and the distal end, the plate further comprising a distal end portion adjacent the distal end that defines at least two circular openings and an intermediate portion disposed between the proximal end and the distal end portion, the intermediate portion defining at least one axially oriented elongated slot;
at least two distal screws for engaging the respective circular openings and a distal end of a bone below a fracture of the bone; and
at least one fastener slidably coupled to the axially oriented elongated slot, the fastener laterally securing the plate adjacent the bone,
wherein a distal end of the elongated slot is defined to be disposed above the fracture of the bone when the plate is secured to the bone with the distal screws.

2. The fracture fixation system of claim 1, wherein the fastener comprises an eyelet slidably coupled to the axially oriented elongated slot and at least one wire for threading through the eyelet and securing around the plate and the bone.

3. The fracture fixation system of claim 2, wherein the at least one axially oriented elongated slot comprises a first slot and a second slot, the slots are axially aligned, the eyelet is slidably coupled to the first slot, and the system further comprises an intermediate screw for engaging the second slot and a portion of the bone above the fracture of the bone, the intermediate screw being axially slidable within the second slot.

4. The fracture fixation system of claim 3, wherein the first and second slots define longitudinal grooves that engage threads on the eyelet and the intermediate screw, respectively.

5. The fracture fixation system of claim 4, wherein the intermediate screw is a bicortical or unicortical screw.

6. The fracture fixation system of claim 5, wherein the distal screws are bicortical locking screws.

7. The fracture fixation system of claim 4, wherein the grooves are oriented at a pitch of greater than 0° relative to the longitudinal axis of the plate for preventing lateral movement of the eyelet and the intermediate screw from the first and second elongated slots, respectively, and allowing the eyelet and the intermediate screw to slide within the first and second elongated slots, respectively.

8. The fracture fixation system of claim 3, wherein the first slot is closer to the proximal end of the plate than the second slot.

9. The fracture fixation system of claim 3, wherein the second slot is closer to the proximal end of the plate than the first slot.

10. The fracture fixation system of claim 1, wherein the fastener comprises an intermediate screw for engaging the slot and a portion of the bone above the fracture of the bone, the intermediate screw being axially slidable within the slot.

11. The fracture fixation system of claim 1, wherein a longitudinal length of the slot is between 2 mm and 10 mm.

12. The fracture fixation system of claim 11, wherein the longitudinal length of the slot is 7 mm.

13. The fracture fixation system of claim 1, wherein the distal screws are bicortical locking screws.

14. The fracture fixation system of claim 1, wherein the bone is a femur.

15. A fracture fixation method comprising:
disposing a plate against a fractured bone, the plate having a proximal end, a distal end, and a longitudinal axis that extends between the proximal end and the distal end, the plate further comprising a distal end portion adjacent the distal end that defines at least two circular openings and an intermediate portion disposed between the proximal end and the distal end portion, the intermediate portion defining at least one axially oriented elongated slot;
engaging at least two distal screws into the respective circular openings and a distal end of the bone below a fracture of the bone;
slidably coupling at least one fastener to the axially oriented elongated slot, the at least one fastener laterally securing the plate adjacent the bone;
wherein a distal end of the elongated slot is disposed above the fracture of the bone.

16. The fracture fixation method of claim 15, wherein slidably coupling the at least one fastener comprises slidably coupling an eyelet to the axially oriented elongated slot, threading at least one wire through the eyelet, and securing the wire around the plate and the bone.

17. The fracture fixation method of claim 16, wherein the at least one axially oriented elongated slot comprises a first slot and a second slot, the slots are axially aligned, the eyelet is slidably coupled to the first slot, and the method further comprises engaging an intermediate screw through the second slot and a portion of the bone above the fracture of the bone, the intermediate screw being axially slidable within the second slot.

18. The fracture fixation method of claim 17, wherein the first and second slots define longitudinal grooves that engage threads on the eyelet and the intermediate screw, respectively.

19. The fracture fixation method of claim 18, wherein the intermediate screw is a bicortical or unicortical screw.

20. The fracture fixation method of claim 19, wherein the distal screws are bicortical locking screws.

21. The fracture fixation method of claim 18, wherein the grooves are oriented at a pitch of between 1° and 2° relative to the longitudinal axis of the plate.

22. The fracture fixation method of claim 17, wherein the first slot is closer to the proximal end of the plate than the second slot.

23. The fracture fixation method of claim 17, wherein the second slot is closer to the proximal end of the plate than the first slot.

24. The fracture fixation method of claim 16, wherein a longitudinal length of the slot is between 2 mm and 10 mm.

25. The fracture fixation method of claim 22, wherein the longitudinal length of the slot is 7 mm.

26. The fracture fixation method of claim 16, wherein the distal screws are bicortical locking screws.

27. The fracture fixation method of claim 15, wherein slidably coupling at least one fastener to the axially oriented elongated slot comprises engaging an intermediate screw through the slot and a portion of the bone above the fracture of the bone, the intermediate screw being axially slidable within the slot.

28. The fracture fixation method of claim 15, wherein the bone is a femur.
